(19) United States
(12) Patent Application Publication
(10) Pub. No.: US 2006/0144737 A1
(43) Pub. Date: Jul. 6, 2006

(54) Storage bag for diapers and baby wipes

(76) Inventor: Christina L. Rein, Flower Mound, TX (US)

Correspondence Address: Philip G. Meyers;Philip G. Meyers Law Office, Suite 302, 1009 Long Prairie Road, Flower Mound, TX, 75022, US

(21) Appl. No.: 11/030,616

(22) Filed: Jan. 6, 2005

(51) Int. Cl. A61B 19/02; B65D 73/00

(52) U.S. Cl. 206/440; 206/494

(57) ABSTRACT

A bag of limited size configured to hold a package of baby wipes and a small number of diapers with little room left over. The bag is small enough to fit inside a purse, yet can be easily found and removed when a diaper change is needed. The invention further provides a diaper changing set which comprises such a bag with a package of baby wipes and at least one diaper.

### TECHNICAL FIELD

The present invention relates generally to infant care products, more particularly to a bag for carrying a limited supply of diapers and baby wipes.

### BACKGROUND OF THE INVENTION

When away from home, a parent or other adult may often be accompanied by an infant in diapers. Frequently, the infant's diaper must be changed while away from home, in surroundings that are not equipped with infant care products. For this reason, most adults having care of an infant while away from home carry such infant care products with them in a diaper bag.

Most diaper bags are designed primarily to store and transport consumable baby care products that are used in caring for the infant while away from home. Many such bags are large enough to also carry a change of clothes, toys, etc. for the infant. Many diaper bags are even large enough to store a rolled-up or folded pad for laying the infant on while changing the infant's diaper. Others open to form a built-in changing pad. These features are attempts to enhance the usefulness of the diaper bag.

The above-mentioned diaper bags do not, however, solve all problems associated with changing an infant's diaper while away from home. In general, improved forms of diaper bags have become larger and more elaborate over time, designed to contain not only diapers and baby wipes, but many other items such as bottles, formula, a change of clothing and the like. Such diaper bags must be sized accordingly, making them less convenient to carry, especially on a short trip or errand where only one or at most two diaper changes are likely to be needed.

Many mothers taking a baby on errands leave the diaper bag at home or in the car and stick a diaper and a small pack of baby wipes in their purse. This eliminates the need to take along a diaper bag, but the diaper and wipes can become separated and difficult to find in the purse, which contains many other objects. The present invention addresses this difficulty.

### SUMMARY OF THE INVENTION

The invention provides a bag of limited size configured to hold a package of baby wipes and a small number of diapers with little room left over. As such, the bag of the invention is small enough to fit inside a purse, yet can be easily found and removed when a diaper change is needed. The invention further provides a system for storing baby accessories which comprises such a bag with a package of baby wipes and at least one diaper contained therein with specific dimensions as further discussed in the detailed description which follows, along with a method of using such a system.

A bag configured for holding a diaper and container of baby wipes according to one aspect of the invention is generally rectangular and includes a front wall, a rear wall, a pair of sides joining the front and rear walls at opposite side edges thereof, a bottom wall and a mouth opposite the bottom wall, which mouth opens onto an internal pocket. The bag when laid flat has a length in the range of 9 to 12 inches, a width in the range of 4 to 8 inches, and can open to provide a mouth and pocket with a cross sectional area in the range of about 5 to 25 in² when at object at least 4 inches wide, such as a disposable diaper or container of baby wipes, is placed in the pocket.

A system for storing baby changing accessories according to another aspect of the invention comprises a generally rectangular bag having a front wall, a rear wall, a pair of sides joining the front and rear walls at opposite side edges thereof, a bottom wall and a mouth opposite the bottom wall, which mouth opens onto an internal pocket. A container of baby wipes is disposed in the pocket and removable from the mouth of the bag. From one to not more than three diapers are stacked on the container of baby wipes in the pocket. The diapers and baby wipes fit closely in the pocket, which may be sized to hold one, two or three diapers plus the container. The diapers plus the flat wipes container fill the pocket in the back with little space left over, but not so tightly that they cannot be easily removed at the time of use.

A method of using the foregoing baby changing system according to another aspect of the invention includes the initial step of storing at least one diaper and container of baby wipes in the bag, then placing the bag in a larger carrying device (such as a purse) containing other items. At the time of use, the bag is removed from the larger carrying device, and then the wipes and a diaper are removed from the bag. The baby is then changed using the diaper and wipes removed from the bag, and any unused wipes and any diapers can then be placed back in the bag after changing the baby. If the bag has a closure flap thereon for closing the mouth of the bag and a closure device for releaseably securing the flap in a closed position, then the closure flap is opened prior to removing the wipes and diaper from the bag. These and other aspects of the invention are further discussed in the detailed description that follows.

### BRIEF DESCRIPTION OF THE DRAWING

In the accompanying drawing, wherein like numerals represent the same or similar elements throughout:

FIG. 1 is a front view of a bag according to the invention, with the flap closed;

FIG. 2 is a front view of the bag of FIG. 1, with the flap open and the inner pocket contents shown in phantom lines;

FIG. 3 is a side view of the bag shown in FIG. 1;

FIG. 4 is a top end view of the bag shown in FIG. 1; and

FIG. 5 is a front view of a purse with a bag according to the invention contained therein shown in phantom lines.

### DETAILED DESCRIPTION

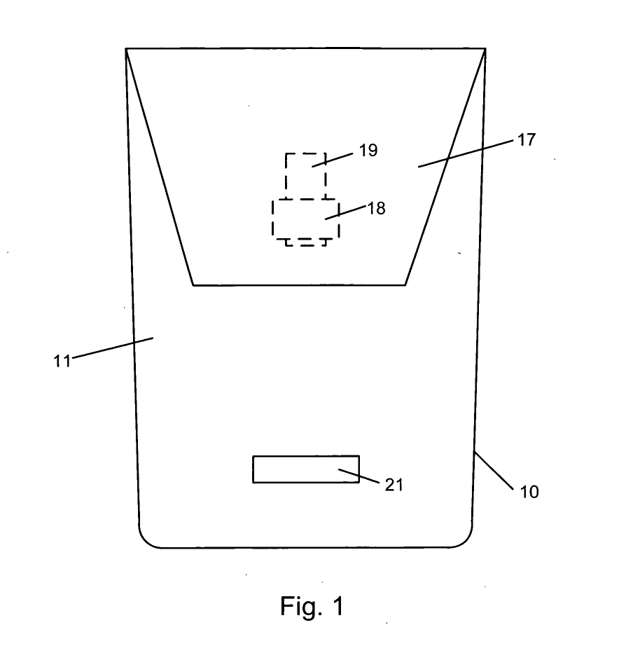

Referring to FIG. 1, a bag 10 according to the invention is preferably made of a soft, flexible durable material such as leather, cloth or fabric. Bag 10 is generally rectangular with a front wall 11 and rear wall 12 sewn together along a pair of side walls 13 and a bottom wall 14. The top end of bag 10 opens in a mouth 16 than can be closed by a fold over flap 17 which is an extension of rear wall 12. Flap 17 can be releasably secured to front wall 11 by means of a suitable fastener, such as Velcro, snaps, a button, magnets, and others commonly used in the industry. In the embodiment shown, a pair of hook and loop elements 18, 19 are sewn to the outside of front wall 11 and the underside of flap 17, respectively. A product label 21 may be placed below the flap as shown, on the flap on the opposite side from the Velcro closure, or at any other suitably prominent location.

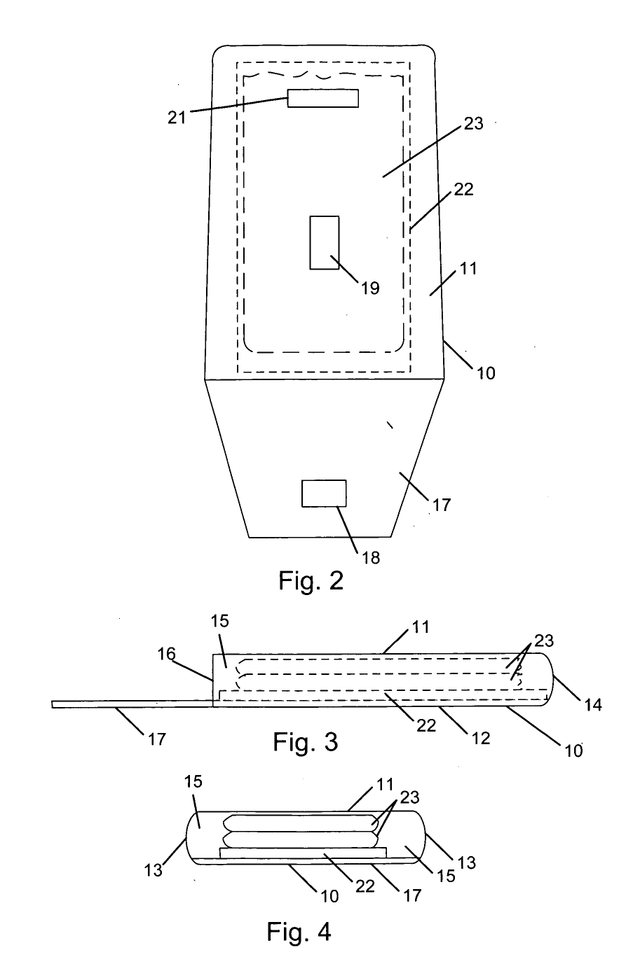

As shown in FIGS. 2-4, the walls of bag 10 are configured so that a rectangular container of baby wipes 22 fits closely inside. Side walls 13 and bottom wall 14 provide bag 10 with sufficient depth so that at least one, preferably from one to three, standard size cloth or disposable diapers 23 also fit inside next to the container 22. However, newborn diapers are smaller than those sold for older babies, such that up to 5 or 6 newborn-size diapers could fit in the bag. This would also be true if only standard size diapers were placed in the bag without a wipes container 22. Container 22 is most commonly a molded plastic compact or clam shell-style of container containing a small number of moistened baby wipes, but could also be a flexible plastic or foil package of baby wipes.

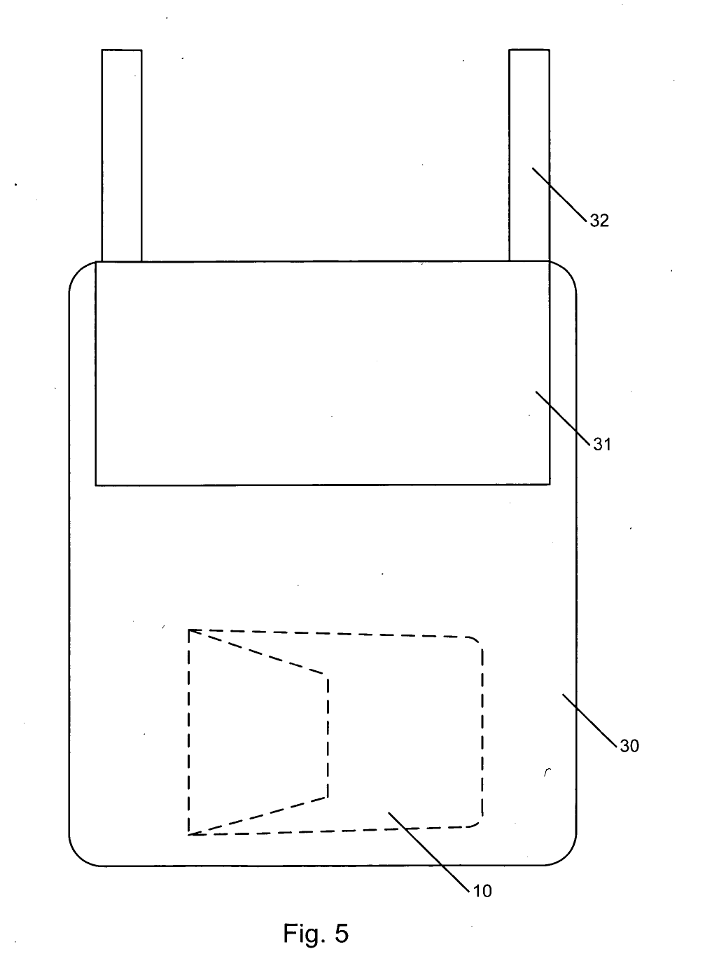

The dimensions of bag 10 are essential to its purpose, namely that it be large enough to contain container 22 and diapers 23 and still fit within a larger purse or handbag 30 having its own closure flap 31 and carrying straps 32 (FIG. 5), but not so large that it requires a handle or strap and has to be carried around separately, like a diaper bag. The width of the bag when laid flat (empty of contents) should be at least 4 inches, preferably 4 to 8 inches and most preferably 5 to 7 inches. The length of the bag when laid flat should be at least 9 inches, preferably 9 to 12 inches and most preferably 9 to 10 inches. Side walls 13 in combination with the widthwise dimension of front and rear walls 11, 12 define the size of an interior pocket 15 and mouth 16. For this purpose, side walls 13 provide a pocket width of at least 1 inch, generally from 2 to 4 inches, most preferably from 2 to 3 inches.

The dimensions of mouth 16 will vary due to the flexible nature of the bag walls. In general, if the resulting pocket 15 is provided with greater depth by side walls 13, then front and rear walls 11, 12 can be narrower, and the converse is also true. A bag of the invention carrying baby wipes in a container and one diaper could, for example, have pocket dimensions of 2″×5.75″, whereas a bag of the invention configured to carry baby wipes and two diapers could have pocket dimensions of 3″×5″. The hard plastic baby wipes container in this example, typical of those commercially available, is about 4.5″ by 8.5″ by less than 1″ thick (0.25″-0.75″ inch thickness is typical). The side walls or “sides” of the bag could also be just seams, and the pocket depth would then be provided by edge portions of the front and rear walls. Hence the size of the pocket and mouth can be defined by its cross-sectional area when in a position to contain an object such as a baby wipes container at least 4 inches wide therein, and may vary from about 5 to 25 in², preferably from about 10 to 20 in².

Bag 10 in the illustrated embodiment tapers slightly towards the bottom as shown for decorative purposes and to use less material, recognizing that the width of pocket 15 is best maximized at mouth 16. The thickness of the fabric or other material used to make bag 10 is not critical to the invention, but should in general be thick enough to be opened and closed many times without failure, but not so thick that is adds unduly to the overall size of the bag. Fabrics ranging from 1/16″ to ⅛″ thick are suitable. Bag 10 can also be made with several different coordinating fabrics, one for the baf body, a different one for the flap and another for the lining. The edge of flap 17 may be provided with a decorative trim or fringe, not shown. Flap 17 preferably extends less than half the length of bag 10 when in its folded-over position as shown in FIG. 1.

Although various embodiments of the invention have been illustrated in the accompanying drawings and described in the foregoing detailed description, it will be understood that the invention is not limited to the embodiments disclosed but, as will be appreciated by those skilled in the art, is susceptible to numerous modifications and variations without departing from the spirit and scope of the invention as hereinafter claimed.

1. A bag configured for holding a diaper and container of baby wipes, comprising a generally rectangular bag body including a front wall, a rear wall, a pair of sides joining the front and rear walls at opposite side edges thereof, a bottom wall and a mouth opposite the bottom wall, which mouth opens onto an internal pocket, wherein the bag when laid flat has a length in the range of 9 to 12 inches, a width in the range of 4 to 8 inches, and can open to provide a mouth and pocket with a cross sectional area in the range of about 5 to 25 in².

2. The bag of claim 1, where the bag is made of a decorative fabric and further comprises:
a closure flap thereon for closing the mouth of the bag, and
a closure device for releasably securing the flap in a closed position.

3. The bag of claim 1, wherein the bag when laid flat has a length in the range of 9 to 10 inches, a width in the range of 5 to 7 inches, and can open to provide a mouth and pocket with a cross sectional area in the range of about 10 to 20 in² when at object about 4 inches wide is placed in the pocket.

4. A system for storing baby changing accessories, comprising:
a generally rectangular bag body including a front wall, a rear wall, a pair of sides joining the front and rear walls at opposite side edges thereof, a bottom wall and a mouth opposite the bottom wall, which mouth opens onto an internal pocket;
a container of baby wipes disposed in the pocket and removable from the mouth of the bag;
and from one to not more than six diapers stacked on the container of baby wipes and disposed in the pocket, wherein the diapers and baby wipes fit closely in the pocket.

5. The system of claim 4, wherein the bag has a closure flap thereon for closing the mouth of the bag, and has a closure device for releasably securing the flap in a closed position.

6. The system of claim 4, wherein the bag when laid flat has a length in the range of 9 to 12 inches, a width in the range of 4 to 8 inches, and can open to provide a mouth and pocket with a cross sectional area in the range of about 5 to 25 in².

7. The system of claim 6, wherein the bag when laid flat has a length in the range of 9 to 10 inches, a width in the range of 5 to 7 inches, and can open to provide a mouth and pocket with a cross sectional area in the range of about 10 to 20 in² when a flat object about 4 inches wide is placed in the pocket.

8. The system of claim 4, wherein from one to not more than three diapers are stacked on the container of baby wipes and disposed in the pocket, wherein the diapers and baby wipes fit closely in the pocket.

9. A method of using a baby changing system which comprises a generally rectangular bag body including a front wall, a rear wall, a pair of sides joining the front and rear walls at opposite side edges thereof, a bottom wall and a mouth opposite the bottom wall, which mouth opens onto an internal pocket, a container of baby wipes disposed in the pocket and removable from the mouth of the bag, and from one to not more than three diapers stacked on the container of baby wipes and disposed in the pocket, wherein the diapers and baby wipes fit closely in the pocket, the method comprising:
storing at least one diaper and container of baby wipes in the bag;
then placing the bag in a large carrying device containing other items;
at time of use, removing the bag from the larger carrying device and then removing the wipes and diaper from the bag; and
changing a baby using the diaper and wipes removed from the bag.

10. The method of claim 9, further comprising replacing the container and any unused diapers back in the bag after changing the baby.

11. The method of claim 9, wherein the bag has a closure flap thereon for closing the mouth of the bag, and has a closure device for releaseably securing the flap in a closed position, further comprising a step of opening the closure flap prior to removing the wipes and diaper from the bag.
